(19) United States
(12) Patent Application Publication
(10) Pub. No.: US 2013/0310339 A1
(43) Pub. Date: Nov. 21, 2013

(54) SEXUAL FUNCTION IMPROVING AGENT

(71) Applicants: Li Han, Chiyoda-ku (JP); Tomoko Tsuji, Chiyoda-ku (JP); Pengtao Li, Beijing (CN); Jun Wang, Beijing (CN); Hongtao Lei, Beijing (CN)

(72) Inventors: Li Han, Chiyoda-ku (JP); Tomoko Tsuji, Chiyoda-ku (JP); Pengtao Li, Beijing (CN); Jun Wang, Beijing (CN); Hongtao Lei, Beijing (CN)

(73) Assignee: NIPPON SUISAN KAISHA, LTD., Chiyoda-ku, Tokyo (JP)

(21) Appl. No.: 13/982,969

(22) PCT Filed: Feb. 1, 2012
(86) PCT No.: PCT/CN12/70815

§ 371 Date: Jul. 31, 2013

(30) Foreign Application Priority Data
Feb. 1, 2011 (CN) PCT/CN2011/070876
Apr. 29, 2011 (CN) PCT/CN2011/073528

(51) Int. Cl. A61K 31/685

(52) U.S. Cl. CPC A61K 31/685
USPC 514/78; 514/77

(57) ABSTRACT

A sexual function improving agent and the use thereof. Said sexual function improving agent comprises a phospholipid including a highly unsaturated fatty acid as a constituent fatty acid.

### TECHNICAL FIELD

The present invention relates to a sexual function improving agent comprising a lipid.

### BACKGROUND

More and more couples, at a rate that has grown to one out of ten couples, are troubled by infertility in conjunction with the move towards marrying late in life in modern society. It is said that in at least 40% of infertility cases, the male has the problem. One major cause is so-called “spermatogenic function disorder”, examples of which include oligozoospermia, oligospermia, asthenospermia, azoospermia and teratospermia. Additionally, infertility is not limited to males of advanced ages, rather a decline in sexual function in adult males on whole has become a social concern. Particularly, in recent years the percentage of people with infertility and diseases or conditions (i.e. metabolic syndrome, stress, etc.) considered to be a factor in decreased sexual function is increasing. Therefore, means for improving infertility and sexual function are needed.

There are several reports associated with sexual function. Japanese Patent Publication No. H10-245340 describes a composition having aphrodisic/invigorating effects including a hot water extract of a cultured mycelium of Cordyceps sinensis as an active ingredient (Patent Document 1). Japanese Patent Publication No. 2004-000171 describes that a functional food including an alcohol extract of maca increases a blood concentration of a growth hormone considered to be effective in suppressing reproductive hypoactivity (Patent Document 2). Japanese Patent Publication No. 2005-306754 describes a composition, or the like, including a benzylic glucosinolate and a benzylic isothiocyanate derived from maca that delivers a male sexual function restorative effect by means of increasing the testosterone level in the blood (Patent Document 3). Japanese Patent Publication No. 2007-112782 describes a composition including a benzylic glucosinolate and/or a benzylic Isothiocyanate dervied from maca that can improve menstrual irregularity and sterility by increasing the concentration of estrogen in the blood (Patent Document 4).

### CITATION LIST

### Patent Document

Patent Document 1: Japanese Patent Publication No. H10-245340

Patent Document 2: Japanese Patent Publication No. 2004-000171

Patent Document 3: Japanese Patent Publication No. 2005-306754

Patent Document 4: Japanese Patent Publication No. 2007-112782

### SUMMARY

### Technical Problem

However, most of the compositions reported hereto and commercially available products are sexual stamina enhancers that are designed to enhance sexual function. A product that suppresses a decline in sexual function or restores declined sexual function from a nutritional function approach by using a substance similar to the components that constitute the body is not known. An object of the present invention is to provide a sexual function improving agent.

### Solution to Problem

In light of the foregoing, the inventors focused on the nutritional functions of lipids, and, as a result of diligent research, discovered that a lipid or phospholipid, for example, a lipid or phospholipid including an n-3 highly unsaturated fatty acid such as eicosapentaenoic acid (EPA) or docosahexenoic acid (DHA) as a constituent fatty acid has a sexual function improving effect. The inventors arrived at the present invention based on this discovery.

The present invention provides the following sexual function improving agents (1) to (12).

(1) A sexual function improving agent comprising a phospholipid as an active ingredient.

(2) A sexual improving agent comprising a phospholipid including a polyunsaturated fatty acid as a constituent fatty acid.

(3) A sexual function improving agent comprising a phospholipid including a highly unsaturated fatty acid as a constituent fatty acid.

(4) The sexual function improving agent according to any one of (1) to (3), wherein the phospholipid consists of a phospholipid including a highly unsaturated fatty acid as a constituent fatty acid.

(5) The sexual function improving agent according to any one of (1) to (4), wherein the highly unsaturated fatty acid is an n-3 highly unsaturated fatty acid.

(6) The sexual function improving agent according to (5), wherein the n-3 highly unsaturated fatty acid is eicosapentaenoic acid or docosahexenoic acid.

(7) The sexual function improving agent according to any one of (1) to (6), wherein the phospholipid is selected from the group consisting of phosphatidylserine, phosphatidyl choline, phosphatidylethanolamine, phosphatidic acid, phosphatidylglycerol, and phosphatidylinositol.

(8) The sexual function improving agent according to any one of (1) to (7) that is used to restore declined sexual function.

(9) The sexual function improving agent according to any one of (1) to (8) that is used to improve reproductive function.

(10) The sexual function improving agent according to any one of (1) to (9), wherein a soybean phospholipid is used as an active ingredient.

(11) The sexual function improving agent according to any one of (1) to (9), wherein purified krill oil is used as an active ingredient.

(12) The sexual function improving agent according to any one of (1) to (11) for administering the phospholipid to a subject at a dosage of 1 to 5000 mg/50 kg BW/day.

The following methods (13) to (18) are provided according to another aspect of the present invention.

(13) A method for improving sexual function comprising orally administering the sexual function improving agent according to any one of (1) to (12) to an animal (14) A method for restoring declined sexual function comprising orally administering the sexual function improving agent according to any one of (1) to (12) to an animal.

(15) A method for improving reproductive function comprising orally administering the sexual function improving agent according to any one of (1) to (12) to an animal.

(16) A method for improving sexual function comprising orally administering the sexual function improving agent according to any one of (1) to (12) to an animal other than a human.

(17) A method for restoring declined sexual function comprising orally administering the sexual function improving agent according to any one of (1) to (12) to an animal other than a human.

(18) A method for improving reproductive function comprising orally administering the sexual function improving agent according to any one of (1) to (12) to an animal other than a human.

The following uses (19) to (21) are provided according to another aspect of the present invention.

(19) A use of a phospholipid in the manufacture of a medicament for improving sexual function.

(20) The use of the phospholipid according to (19), wherein the improvement in sexual function restores declined sexual function.

(21) The use of the phospholipid according to (19) or (20), wherein the improvement in sexual function improves reproductive function.

The following food, animal feed, or pharmaceutical preparation of (22) is provided according to another aspect of the present invention.

(22) A food, an animal feed, or a pharmaceutical preparation comprising the sexual function improving agent according to any one of (1) to (12).

The following sexual function improving agents (23) to (29) are provided according to another aspect of the present invention.

(23) A sexual function improving agent comprising a soybean phospholipid as an active ingredient.

(24) A sexual function improving agent comprising krill oil as an active ingredient.

(25) The sexual function improving agent according to (24), wherein the krill oil is purified krill oil.

(26) The sexual function improving agent according to (24) or (25), wherein the krill oil is purified via a thermal coagulum of krill.

(27) The sexual function improving agent according to any one of (23) to (26) that is used to restore declined sexual function.

(28) The sexual function improving agent according to any one of (23) to (27) that is used to improve reproductive function.

(29) The sexual function improving agent according to any one of (23) to (28) for administering the phospholipid or krill oil to a subject at a dosage of 1 to 5,000 mg/50 kg BW/day.

The following methods of (30) to (35) are provided according to another aspect of the present invention.

(30) A method for improving sexual function comprising orally administering the sexual function improving agent according to any one of (23) to (29) to an animal (31) A method for restoring declined sexual function comprising orally administering the sexual function improving agent according to any one of (23) to (29) to an animal.

(32) A method for improving reproductive function comprising orally administering the sexual function improving agent according to any one of (23) to (29) to an animal.

(33) A method for improving sexual function comprising orally administering the sexual function improving agent according to any one of (23) to (29) to an animal other than a human.

(34) A method for restoring declined sexual function comprising orally administering the sexual function improving agent according to any one of (23) to (29) to an animal other than a human.

(35) A method for improving reproductive function comprising orally administering the sexual function improving agent according to any one of (23) to (29) to an animal other than a human

The following uses (36) to (39) are provided according to another aspect of the present invention.

(36) A use of a soybean phospholipid in the manufacture of a medicament for improving sexual function.

(37) A use of krill oil in the manufacture of a medicament for improving sexual function.

(38) The use according to (36) or (37), wherein the improvement in sexual function restores declined sexual function.

(39) The use according to any one of (36) to (38), wherein the improvement in sexual function is improvement of reproductive function.

The following food, animal feed, or pharmaceutical preparation of (40) is provided according to another aspect of the present invention.

(40) A food, an animal feed, or a pharmaceutical preparation comprising the sexual function improving agent according to any one of (23) to (29).

### Advantageous Effects of Invention

According to the present invention, sexual function in animals is improved. Particularly, the present invention is beneficial in restoring sexual function that has declined due to aging and/or suppressing a decline in sexual function due to aging. Furthermore, the present invention is beneficial in improving reproductive function due to aging. It is shown that the present invention exerts its effect without affecting sex hormone concentration in blood. Such characteristics vary greatly from those of existing sexual stamina enhancers that have invigorating effects.

### BRIEF DESCRIPTION OF THE DRAWINGS

FIG. 1 is a chart showing results of measuring a incubation period of mounting (mount latency) of each administration group of Test Example 1. Cont.: Male animals having declined function and advanced age to which a sexual function improving agent is not administered; Base: Young animals having normal sexual function; K-PSL: K-PS low dose group, K-PSM: K-PS middle dose group, K-PSH: K-PS high dose group; K-PCL: K-PC low dose group, K-PCM: K-PC middle dose group, K-PCH: K-PC high dose group.

FIG. 2 is a chart showing results of measuring a mounting frequency (mount frequency) of each administration group of Test Example 1. Cont.: Male animals having declined function and advanced age to which a sexual function improving agent is not administered; Base: Young animals having normal sexual function; K-PSL: K-PS low dose group, K-PSM: K-PS middle dose group, K-PSH: K-PS high dose group; K-PCL: K-PC low dose group, K-PCM: K-PC middle dose group, K-PCH: K-PC high dose group.

FIG. 3 is a chart showing results of measuring an incubation period of copulation (intromission latency) of each administration group of Test Example 1. Cont.: Male animals having declined function and advanced age to which a sexual function improving agent is not administered; Base: Young animals having normal sexual function; K-PSL: K-PS low dose group, K-PSM: K-PS middle dose group, K-PSH: K-PS high dose group; K-PCL: K-PC low dose group, K-PCM: K-PC middle dose group, K-PCH: K-PC high dose group.

FIG. 4 is a chart showing results of measuring a copulation frequency (intromission frequency) of each administration group of Test Example 1. Cont.: Male animals having declined function and advanced age to which a sexual function improving agent is not administered; Base: Young animals having normal sexual function; K-PSL: K-PS low dose group, K-PSM: K-PS middle dose group, K-PSH: K-PS high dose group; K-PCL: K-PC low dose group, K-PCM: K-PC middle dose group, K-PCH: K-PC high dose group.

FIG. 5 is a chart showing results of calculating a copulation ratio (intromission efficiency) of each administration group of Test Example 1. Cont.: Male animals having declined function and advanced age to which a sexual function improving agent is not administered; Base: Young animals having normal sexual function; K-PSL: K-PS low dose group, K-PSM: K-PS middle dose group, K-PSH: K-PS high dose group; K-PCL: K-PC low dose group, K-PCM: K-PC middle dose group, K-PCH: K-PC high dose group.

FIG. 6 is a chart showing results of measuring a weight of the testis (+the epididymis) of each administration group of Test Example 1. Cont.: Male animals of reduced function advanced age to which a sexual function improving agent is not administered; K-PSL: K-PS low dose group, K-PSM: K-PS middle dose group, K-PSH: K-PS high dose group; K-PCL: K-PC low dose group, K-PCM: K-PC middle dose group, K-PCH: K-PC high dose group.

FIG. 7 is a chart showing results of measuring a weight of the seminal vesicles (+the prostate) of each administration group of Test Example 1. Cont.: Male animals of reduced function and advanced age to which a sexual function improving agent is not administered: K-PSL: K-PS low dose group, K-PSM: K-PS middle dose group, K-PSH: K-PS high dose group; K-PCL: K-PC low dose group, K-PCM: K-PC middle dose group, K-PCH: K-PC high dose group.

FIG. 8 is a chart showing pathological sections of spermary (testis), especially convoluted seminiferous tubules, of Test Example 2. (A) Normal group (Base), (B) Advanced age group (Control), (C) K-PC low dose group (K-PCL), (D) K-PC middle dose group (K-PCM), (E) K-PC high dose group (K-PCH).

FIG. 9 is a chart showing thickness of convoluted seminiferous tubules obtained from mice in each group of Test Example 2. Base: Normal group, Cont.: Advanced age group, K-PCL: K-PC low dose group, K-PCM: K-PC middle dose group, K-PCH: K-PC high dose group.

FIG. 10 is a chart showing total count of sperm obtained from mice in each group of Test Example 3. Base: Normal group, Cont.: Advanced age group, K-PCL: K-PC low dose group, K-PCM: K-PC middle dose group.

FIG. 11 is a chart showing motile percent of sperm obtained from mice in each group of Test Example 3. Base: Normal group, Cont.: Advanced age group, K-PCL: K-PC low dose group, K-PCM: K-PC middle dose group.

FIG. 12 is a chart showing progressive percent of sperm obtained from mice in each group of Test Example 3. Base: Normal group, Cont.: Advanced age group, K-PCL: K-PC low dose group, K-PCM: K-PC middle dose group.

FIG. 13 is a chart showing weight of parorchis obtained from mice in each group of Test Example 3. Cont.: Advanced age group, K-PCL: K-PC low dose group, K-PCM: K-PC middle dose group.

FIG. 14 is a chart showing weight of prostate obtained from mice in each group of Test Example 3. Cont.: Advanced age group, K-PCL: K-PC low dose group, K-PCM: K-PC middle dose group.

FIG. 15 is a chart showing comparison of the Inhibin B concentration in blood of each administration group in Test Example 4. Base: Normal group, Cont.: Advanced age group, K-PSH: K-PS high dose group, K-PSM: K-PS middle dose group, K-PSL: K-PS low dose group, K-PCH: K-PC high dose group, K-PCM: K-PC middle dose group; K-PCL: K-PC low dose group.

FIG. 16 is a chart showing comparison of the FSH (Follicle-Stimulating Hormone) concentration in blood of each administration group in Test Example 4. Base: Normal group, Cont.: Advanced age group, K-PSH: K-PS high dose group,

K-PSM: K-PS middle dose group, K-PSL: K-PS low dose group, K-PCH: K-PC high dose group, K-PCM: K-PC middle dose group; K-PCL: K-PC low dose group.

FIG. 17 is a chart showing comparison of the total count of sperms of each administration group in Test Example 5. Cont.: Advanced age group, F-Oil: fish oil, S-PC: soybean PC, F+S: fish oil plus soybean PC.

FIG. 18 is a chart showing comparison of the motile percent of sperms of each administration group in Test Example 5. Cont.: Advanced age group, F-Oil: fish oil, S-PC: soybean PC, F+S: fish oil plus soybean PC.

FIG. 19 is a chart showing comparison of the progress percent of sperms of each administration group in Test Example 5. Cont.: Advanced age group, F-Oil: fish oil, S-PC: soybean PC, F+S: fish oil plus soybean PC.

FIG. 20 is a chart showing comparison of the weight of testis of each administration group in Test Example 5. Cont.: Advanced age group, F-Oil: fish oil, S-PC: soybean PC, F+S: fish oil plus soybean PC.

FIG. 21 is a chart showing comparison of the weight of epididymis of each administration group in Test Example 5. Cont.: Advanced age group, F-Oil: fish oil, S-PC: soybean PC, F+S: fish oil plus soybean PC.

### DESCRIPTION OF EMBODIMENTS

Hereinafter, the present invention is described in more detail.

The present invention provides a sexual function improving agent comprising a phospholipid as an active ingredient. And, the present invention provides a sexual function improving agent comprising a phospholipid including a polyunsaturated fatty acid as a constituent fatty acid as an active ingredient. Furthermore, the present invention provides a sexual function improving agent comprising a lipid including a highly unsaturated fatty acid as a constituent fatty acid as an active ingredient. The sexual function improving agent of the present invention may comprise a component of soybean or krill origin comprising a lipid such as, for example, a ground product of a krill, a krill meal, krill meat, or the like.

The sexual function improving agent of the present invention comprises an effective amount of the phospholipid or the lipid. Here, “effective amount” refers to an amount needed to improve sexual function, and is for example, from 1 to 5,000 mg/l kg of body weight, preferably from 2.5 to 2,500 mg/l kg of body weight, and particularly preferably from 10 to 1,000 mg/l kg of body weight of an animal per day. Especially in cases of human adults, the effective amount is from 1 to 10,000 mg/50 kg of body weight, preferably from 2.5 to 5,000 mg/50 kg of body weight, more preferably from 5 to 3000 mg/kg of body weight, and particularly preferably from 10 to 1,000 mg/50 kg of body weight per day. In cases of human adults, it is preferable that a greater amount of the lipid be ingested to achieve more prominent sexual function improvement effects, but if too great, undesirable characteristics such as the sexual function improving agent becoming excessively oily, absorption lagging, dyspepsia, indigestion, loss of appetite, and the like will occur. These ingestion amounts may be an amount ingested at one time or may be an amount ingested multiple times, for example, two or three times.

Lipids, along with proteins, nucleic acids and saccharides (carbohydrates), are a major structural component of living organisms. And in all biological systems, lipids are an important class of compounds that not only serve as a source of energy but also constitute the membrane structure of cells which are fundamental units of life. As stated, the lipid is a component vital to an organism, and comprises an ester bond between an alcohol and a fatty acid. Other than straight chain alcohols, examples of the alcohol include glycerol (glycerin), sterol, and the like. Examples of the fatty acid include various saturated fatty acids or unsaturated fatty acids. Of the lipids, those that have ester bonds between a hydroxyl group of the glycerol, as the alcohol, and a carboxyl group of the fatty acid are referred to as “biological lipids”. Examples of the biological lipids include glycerides and phospholipids.

Examples of the glycerides include triacylglycerols (triglycerides), where all three hydroxyl groups of the glycerol are ester bonded with the fatty acid; diacylglycerols (diglycerides), where two of the three hydroxyl groups of the glycerol are ester bonded with the fatty acid and the other one hydroxyl group is left as-is; and monoacylglycerols (monoglycerides), where one of the three hydroxyl groups of the glycerol is ester bonded with the fatty acid and the other two hydroxyl groups are left as-is.

“Phospholipid” refers to a substance in which at least one of the three hydroxyl groups of the glycerol is ester bonded with the fatty acid and the other one hydroxyl group is covalently bonded with a phosphate. The phosphates ordinarily covalently bond with the first or the third hydroxyl group of the glycerol. Amounts of the triacylglycerol and the phospholipid as the biological lipid are great and are important.

Phospholipids are known as major components constituting cell membranes and have a hydrophilic phosphate part and a hydrophobic fatty acid part. Phospholipids are divided into diacylglycerophospholipids having the fatty acid parts at a first position and second position of the glycerol backbone and lysoacylglycerophospholipids. Lysoacylglycerophospholipids are divided into 1-acylglycerophospholipids having the fatty acid part only at the first position on the glycerol backbone, and 2-acylglycerophospholipids having the fatty acid part only at the second position of the glycerol backbone. In the present specification, “phospholipid” includes all of these, but the diacylglycerophospholipid is particularly preferable. Examples of the diacylglycerophospholipid include phosphatidyl choline (PC), phosphatidylethanolamine (PE), phosphatidylserine (PS), phosphatidylinositol (PI), phosphatidylglycerol (PG), cardiolipin (CL), phosphatidic acid (PA), and mixtures of two or more thereof; preferably PC, PE, PS, PI, PA, and mixtures of two or more of thereof; and particularly preferably PC, PS, or a mixture thereof Examples of the lysoacylglycerophospholipid include 1- or 2-lyso PC, 1- or 2- lyso PE, 1- or 2-lyso PS, 1- or 2-lyso PI, 1- or 2-lyso PG, 1- or 2-lyso CL, 1- or 2-lyso PA, and mixtures of two or more thereof; preferably 1- or 2-lyso PC, 1- or 2-lyso PE, 1- or 2-lyso PS, 1- or 2-lyso PI, 1- or 2-lyso PA, and mixtures of two or more thereof; and particularly preferably 1- or 2-lyso PC, 1- or 2-lyso PS, and a mixture thereof.

The lipid according to the present invention has a polyunsaturated fatty acid as the fatty acid part. In the present specification, “polyunsaturated fatty acid” refers to a fatty acid having two or more double bonds. The scope of the polyunsaturated fatty acid encompasses linoleic acid (18:2, LA) and a highly unsaturated fatty acid.

The lipid according to the present invention has a highly unsaturated fatty acid as the fatty acid part. In the present specification, “highly unsaturated fatty acid” refers to a fatty acid having three or more double bonds and a carbon number of 18 or higher, and preferably 20 or higher. An n-3 highly unsaturated fatty acid is preferable as the highly unsaturated fatty acid. In the present specification, “n-3 highly unsaturated fatty acid” refers to a fatty acid wherein the third and fourth carbons, counting from the terminal carbon opposite the carboxyl side of the fatty acid molecule, are double bonded. Examples of such a fatty acid include α-linolenic acid (18:3, ALA), eicosapentaenoic acid (20:5, EPA), docosapentaenoic acid (22:5, DPA), docosahexenoic acid (22:6, DHA), and the like, preferably EPA and DHA. A percentage of the n-3 highly unsaturated fatty acid occupying the constituent fatty acid of the lipid of the present invention as a fatty acid composition ratio is, for example, from 1 to 100%, preferably from 10 to 90%, and more preferably from 20 to 80%. Because fluidity of the n-3 highly unsaturated fatty acid is high, as greater amounts are included in the lipid greater effectiveness in providing more beneficial physical characteristics at low temperatures will be achieved. However, at best, un-purified natural materials only contain about 60% of the n-3 highly unsaturated fatty acid and attempting to increase a concentration thereof leads to added costs due to concentration.

Furthermore, an n-6 highly unsaturated fatty acid is preferable as the highly unsaturated fatty acid. In the present specification, “n-6 highly unsaturated fatty acid” refers to a fatty acid wherein the sixth and seventh carbons, counting from the terminal carbon opposite the carboxyl side of the fatty acid molecule, are double bonded. Examples of such a fatty acid include γ-linolenic acid (18:3, GLA), dihomo-γ-linolenic acid (20:3, DGLA), arachidonic acid (20:4, AA), and the like, preferably GLA and AA. A percentage of the n-6 highly unsaturated fatty acid occupying the constituent fatty acid of the lipid of the present invention as a fatty acid composition ratio is, for example, from 1 to 100%, preferably from 10 to 90%, and more preferably from 20 to 80%. Because fluidity of the n-6 highly unsaturated fatty acid is high, as greater amounts are included in the lipid greater effectiveness in providing more beneficial physical characteristics at low temperatures will be achieved.

Any material comprising a lipid such as those described above can be used as the lipid of the present invention. Examples of such a material include fish and shellfish extracts, animal extracts, egg yolk extract, plant extracts, fungi extracts, and the like, specifically, krill oil, fish oil, fish extract, squid extract, bonito ovary extract, animal extract or egg yolk extracts of an animal given a feed compounded with n-3 highly unsaturated fatty acid, soy extract, flaxseed oil, extracts of genetically modified plants, and the like, and extracts and the like of labyrinthulea. Examples of materials that include a particularly large amount of the lipid include krill oil, squid extract, bonito ovary extract, and soy extract. By using concentrating, extracting and/or purifying, compounding and other techniques known conventionally in the art, a lipid concentration in these materials and a purity can be regulated as desired. For example, by appropriately compounding krill oil, fish oil, flaxseed oil, soy oil, or perilla oil containing the highly unsaturated fatty acid; and krill oil, plant oil (phospholipid of soy origin, phospholipid of rapeseed origin), animal extract (phospholipid of egg yolk origin), marine extract (phospholipid of squid extract origin, phospholipid of fish extract origin, phospholipid of krill origin), or the like containing the lipid, a lipid including both the highly unsaturated fatty acid and the lipid at high concentrations can be produced.

Generally, if the orally ingested lipid is triacylglycerol or diacylglycerol the lipid is hydrolyzed into a free fatty acid and a monoacylglycerol or, in the case of the phospholipid, into a free fatty acid and a lysoacylglycerophospholipid, a phosphatidic acid, or a lysophosphatidic acid by gastric lipase and pancreatic lipase. These hydrolyzates are dissolved by bile acid and by the forming of bile acid micelles. Small intestine epithelial cells incorporate the hydrolyzates from the bile acid micelles and triacylglycerols and diacylglycerophospholipids are resynthesized from the incorporated hydrolyzates. Thus, when the free highly unsaturated fatty are acids ingested by an organism, they are incorporated into the small intestine epithelial cells via bile acid and micelle formation and bond with the glycerol and/or phosphates in the organism. Thereby, they are incorporated as constituent fatty acids of triacylglycerols and/or diacylglycerophospholipids. Therefore, by ingesting the phospholipid or the triacylglycerol together with the highly unsaturated fatty acid, the percentage of phospholipids including highly unsaturated fatty acids among the phospholipids or the triacylglycerol resynthesized in the organism can be increased, and a greater sexual function improvement effect can be obtained.

For example, when ingesting the phospholipid together with the highly unsaturated fatty acid, a lipid that is appropriately compounded with a fat/oil including both may be used. Alternately, a phospholipid including a highly unsaturated fatty acid as a constituent fatty acid may be used. From the perspectives of ease of absorption, substance stability, and ease of quality control the phospholipid including the highly unsaturated fatty acid as the constituent fatty acid is particularly preferable. For example, when ingesting the triacylglycerol together with the highly unsaturated fatty acid, a lipid that is appropriately compounded with a fat/oil including both may be used. Alternately, a triacylglycerol including a highly unsaturated fatty acid as a constituent fatty acid may be used. From the perspectives of ease of absorption, substance stability, and ease of quality control the triacylglycerol including the highly unsaturated fatty acid as the constituent fatty acid is particularly preferable.

The sexual function improving agent of the present invention may also include other components included in krill oil, such as, for example, astaxanthin, sterol, and the like. Astaxanthin is a compound belonging to carotenoids commonly found in crustacea such as crabs and shrimp. The astaxanthin may be present in a free state or may be present in a lipid state via ester bonding. Additionally, from 1 to 10,000 ppm, preferably from 5 to 5,000 ppm, and more preferably from 10 to 1,000 ppm of the astaxanthin in a free state may be separately added to the sexual function improving agent. The astaxanthin, as an endogenous antioxidant, contributes to the stability of the highly unsaturated fatty acid, and, thus, is preferably included in abundance. However, if too much of the astaxanthin is included, problems with color and taste will easily occur.

The sterol contributes to the fluidity of the lipid and also contributes to the absorption of the sexual function improving agent of the present invention.

In the present specification, it is sufficient that the “krill” be an arthropod belonging to the phylum Arthropoda, subphylum Crustacea, class Malacostraca and includes arthropods belonging to the phylum Arthropoda, subphylum Crustacea, class Malacostraca, order Eucarida, family Euphausiacea such as, for example, Euphausia superba, and arthropods belonging to the phylum Arthropoda, subphylum Crustacea, class Malacostraca, order Euphausiacea, family Euphausiidae such as, for example, Mysidacea caught in the seas around Japan, and the like. However, from the perspective of stability of catch volume and uniformity of the lipid component, Antarctic krill are particularly preferable. In the present specification, “lipid of krill origin” refers to a lipid obtained from the krill described above.

The lipid of krill origin used in the present invention can be acquired by a known method of manufacturing. For example, the phospholipid can be produced while referring to the known methods described in WO2000/023546A1, WO2009/027692A1, WO2010/035749A1, WO2010/035750A1, or the like. At the least, the lipid that can be produced via the methods described in the international publications can be preferably used in the sexual function improving agent of the present invention.

The sexual function improving agent of the present invention can be obtained by, for example, following a method described in the international publications mentioned above, and using an appropriate organic solvent to extract the PC from a solid content originating from a source material of krill. Appropriate examples of the organic solvent include alcohols such as methanol, ethanol, propanol, isopropanol, butanol, propylene glycol, butylene glycol; methyl acetate, ethyl acetate, acetone, chloroform, toluene, pentane, hexane, cyclohexane, and the like. These may be used alone or in combinations of two or more. In such cases, a mixture ratio of the solvent or a ratio of the source material to the solvent can be set as desired.

The solid content originating from a source material of krill can be obtained, for examples, by obtaining a squeezed fluid by squeezing all or a part of dried, milled, raw, or frozen krill; and separating the solid content and a water soluble component by heating the squeezed fluid. For the squeezing, a commonly used apparatus can be used. For example, a hydraulic press, a screw press, a meat and bone separator, a press dehydrator, a centrifuge, and the like, or a combination thereof can be used.

The squeezed fluid may be heated under atmospheric pressure, pressurized, or reduced pressure conditions to 50° C. or higher, and preferably to from 70 to 150° C., and particularly preferably to from 85 to 110° C. Through this heating, the solid content (thermal coagulum) and the water soluble component are separated, and through filtering, centrifuging, or the like, a thermal coagulum is obtained. Furthermore, the thermal coagulum can be appropriately dried and used. The drying can be performed by any one or a combination of hot air drying, drying using steam, drying by high frequency/microwave heating, vacuum/reduced pressure drying, drying by freezing and thawing, and drying using a drying agent. When drying, if the temperature is too high, the oxidized lipid will emit a foul odor. Therefore, it is beneficial that the drying be performed at 90° C. or lower, preferably at 75° C. or lower, and more preferably at 55° C. or lower. Drying is preferable because volatile impurities are removed thereby. The thermal coagulum or the dried product thereof comprises astaxanthin, and therefore can be preferably used as the sexual function improving agent of the present invention.

Additionally, a purification method wherein an amount of residue of the impurities is small is preferred. The thermal coagulum of the krill squeezed fluid or the dried product thereof is fit for such a purpose because a concentration of the water soluble component thereof can be reduced by washing with water. The washing with water can be performed using an amount of freshwater or saltwater 4-times, and preferably 10-times the amount of the dry content weight in the thermal coagulum or the dried product thereof The washing is preferably performed two times or more, and more preferably 3 times or more. The washing with water can be performed by adding water to a container in which the thermal coagulum or the dried product thereof has been placed, and then separating the moisture content after waiting for 5 minutes or longer. Depending on a shape of the thermal coagulum or the dried product thereof, a sufficient amount of agitation can also be effective. Additionally, the washing with water can be performed by washing the thermal coagulum or the dried product thereof in a container with running water.

Furthermore, for example, by treating the thermal coagulum or the dried product thereof, or a washed product thereof as described below, a fraction including a greater amount of the PC can be obtained. For example, an extract oil is obtained by treating the thermal coagulum or the dried product thereof, or the washed product thereof with a solvent such as ethanol, hexane, chloroform, acetone, or the like. Next, impurities and the phospholipid fraction are separated by subjecting the extract oil to chromatography using silica gel or the like, and the phospholipid fraction is concentrated. The fraction is rich in PC.

The PS can be obtained by extraction from animal tissue, but can be more effectively obtained by using a different phospholipid as a starting material. For example, the PS can be obtained by enzymatically reacting PC and serine using the catalytic action of phospholipase D. An amount of the serine used with respect to an amount of the phospholipid used in the reaction can be set to from 0.5 to 3.0 weight ratio, and preferably from 1.0 to 2.0 weight ratio. The phospholipase D can be used at from 0.05 to 0.2 weight ratio, and preferably from 0.1 to 0.15 weight ratio per 1 g of the phospholipid. The phospholipase D that can be used include those originating from microorganisms and vegetables such as cabbage and the like.

The enzymatic reaction can be performed using a method known in the art. For example, the enzymatic reaction can be performed in a solvent such as ethyl acetate and the like at from 35 to 45° C. for from 20 to 24 hours.

In the present invention, the scope of a sexual function encompasses sex activity and reproductive function. The sex activity refers to for example, reproductive behavior such as frequency or duration of mounting behavior, copulation behavior, and the like. The reproductive function refers to a sexual function other than the sex activity, for example, functions relating to fertility such as number of sperm, motility of sperm, ability of a primary spermatocyte for differentiation to a sperm, and the like.

Unless otherwise stipulated, evaluations of the sexual function improvement effects achieved by means of the sexual function improving agent of the present invention are performed based on the results of testing of the following items.

1. Incubation period of mounting: Time required to perform first mounting after the start of the mating test

2. Incubation period of copulation (ejaculation): Time required to perform first copulation after the start of the mating test

3. Mounting frequency: Number of times of mounting performed during a duration of the test

4. Copulation frequency: Number of times of copulation performed during a duration of the test

5. Ratio of mounting frequency to copulation frequency (copulation ratio): The number of times of copulation divided by the number of times of mounting

6. Weight of the testis (+the epididymis) and the seminal vesicles (+the prostate)

7. Evaluation on the state of sperm such as total count of sperm, motile percent of sperm, progressive percent of sperm, path velocity, and progressive velocity

8. Sex hormone concentration in serum

The sexual function improving agent is administered to a male animal (test animal), and, after administration, testing of the items described above is performed.

The obtained test results were compared with test results of male animals of reduced function and advanced age to which a sexual function improving agent is not administered (control) and young animals having normal sexual function (base) as comparison subjects and the effectiveness of the sexual function improving agent was evaluated based on the following criteria.
In cases where the results of the test animal exceed the control but are not significantly different from the base or are lower that the base, the sexual function improving agent is evaluated to have a sexual function improvement effect.
In cases where the results of the test animal exceed the control and are significantly different from the base or exceed the base, the sexual function improving agent is evaluated to have a sexual function invigorating effect.
In cases where the results of the test animal are not significantly different from the control or are lower that the control, the sexual function improving agent is evaluated to not have a sexual function improvement effect.

With the male animals to which the sexual function improving agent of the present invention was administered, in comparison to the male animals of declined function and advanced age to which a sexual function improving agent was not administered, a reduction in the incubation period of mounting and an increase in mounting frequency, a reduction in the incubation period of copulation and an increase in the copulation frequency, and an increase in the copulation ratio were observed. Thus, the present invention can provide a method for improving sexual function, comprising administration of the sexual function improving agent to a male animal such as a human or the like.

Furthermore, because the sexual function improving agent of the present invention increases the weight of the reproductive organs of the male animal to which it is administered, particularly the seminal vesicles (+the prostate), it is suggested that spermatogenesis is activated. Thus, the present invention can provide a method for activating spermatogenesis, comprising administration of the sexual function improving agent to a male animal such as a human or the like.

Animals, other than “humans” as referred to in this specification, to which the sexual function improving agent of the present invention can be applied as animal or fish feed or include domestic animals such as cows, horses, pigs, sheep, goats, mules, camels, llamas, Asian elephants, alpacas, reindeer (caribou), zebras (zebu), water buffalo, yaks, guinea pigs, hares (rabbits), minks, chicken, ducks, geese, turkeys, Muscovy ducks, quails, ostriches, feral pigeons, pheasants, cormorants, dogs, cats, hamsters, guinea pigs, ferrets, squirrels, monkeys and the like; fish: sea bream, tuna, rudderfish, goldstriped amberjack, jack mackerel, chub mackerel, Japanese seaperch, eel, halibut, olive flounder, globefish, salmon, trout, catfish, sea bass, barramundi, cobia, tilapia, and the like.

The sexual function improving agent of the present invention may be mixed with a component having conventionally known invigorating effects as necessary such as, for example, of maca, zinc, viper extract, ginseng, cistanchis herba, or the like and used. The sexual function improving agent of the present invention may be mixed with components such as conventionally known colorants, preservatives, perfumes, flavorants, coating agents, antioxidants, vitamins, amino acids, peptides, proteins, minerals (i.e. iron, zinc, magnesium, iodine, etc.) and the like.

Examples of the antioxidant includes tocopherol, dried yeast, glutathione, lipoic acid, quercetin, catechin, coenzyme Q10, enzogenol, proanthocyanidins, anthocyanidin, anthocyanin, carotenes, lycopene, flavonoid, resveratrol, isoflavones, zinc, melatonin, ginkgo leaf, Alpinia zerumbet leaf, hibiscus, or extracts thereof

Examples of the vitamin include the vitamin A group (i.e. retinal, retinol, retinoic acid, carotene, dehydroretinal, lycopene, and salts thereof); the vitamin B group (i.e. thiamin, thiamin disulfide, dicethiamine, octotiamine, cycotiamine, bisibuthiamine, bisbentiamine, prosultiamine, benfotiamine, fursultiamine, riboflavin, flavin adenine dinucleotide, pyridoxine, pyridoxal, hydroxocobalamin, cyanocobalamin, methylcobalamin, deoxyadenocobalamin, folic acid, tetrahydro folic acid, dihydro folic acid, nicotinic acid, nicotinic acid amide, nicotinic alcohol, pantothenic acid, panthenol, biotin, choline, inositol, pangamic acid, and salts thereof); the vitamin C group (i.e. ascorbic acid and derivatives thereof, erythorbic acid and derivatives thereof, and salts thereof that are pharmacologically acceptable); the vitamin D group (i.e. ergocalciferol, cholecalciferol, hydroxycholecalciferol, dihydroxycholecalciferol, dihydrotachysterol, and salts thereof that are pharmacologically acceptable); the vitamin E group (i.e. tocopherol and derivatives thereof, ubiquinone derivatives, and salts thereof that are pharmacologically acceptable); and other vitamins (i.e. carnitine, ferulic acid, γ-oryzanol, orotic acid, rutin (vitamin P), eriocitrin, hesperidin, and salts thereof that are pharmacologically acceptable).

Examples of the amino acid include leucine, isoleucine, valine, methionine, threonine, alanine, phenylalanine, tryptophan, lysine, glycine, asparagine, aspartic acid, serine, glutamine, glutamic acid, proline, tyrosine, cysteine, histidine, ornithine, hydroxyproline, hydroxylysine, glycylglycine, aminoethylsulfonic acid (taurine), cystine, and salts thereof that are pharmacologically acceptable.

The sexual function improving agent of the present invention may be prepared in the form of a pharmaceutical composition, functional food, health food, supplement, or the like such as, for example, various solid formulations such as granule formulations (including dry syrups), capsule formulations (soft capsules and hard capsules), tablet formulations (including chewable tablets and the like), powdered formulations (powders), pill formulations, and the like; or liquid formulations such as liquid formulations for internal use (including liquid formulations, suspension formulations, syrup formulations, etc.) and the like.

Examples of additives that help with formulation include excipients, lubricants, binders, disintegrating agents, fluidization agents, dispersing agents, wetting agents, preservatives, thickening agents, pH adjusting agents, colorants, corrigents, surfactants, and solubilization agents. Additionally, prepared as a liquid formulation, thickening agents such as pectin, xanthan gum, guar gum, and the like can be compounded. Moreover, the sexual function improving agent of the present invention can be formed into a coated tablet formulation by using a coating agent, or be formed into a paste-like gelatin formulation. Furthermore, even when preparing the sexual function improving agent in other forms, it is sufficient to follow conventional methods.

Furthermore, the sexual function improving agent of the present invention can be used as various foods and drinks such as, for example, beverages, confectioneries, breads, soups, and the like, or as an added component thereof Methods of manufacturing these foods and drinks are not particularly limited as long as the effectiveness of the present invention is not hindered, and it is sufficient that a process used by a person skilled in the art for each application be followed.

Furthermore, the sexual function improving agent of the present invention can be used as a feed for animals, other than humans, or as an added component thereof The sexual function improving agent of the present invention may be compounded with the animal feed that is normally administered to each animal, and can be used regardless of the nature of the animal feed, be it mash, flakes, crumble, powder, granules, moist pellets, dry pellets, EP pellets, or the like. Methods of manufacturing these animal feeds are not particularly limited as long as the effectiveness of the present invention is not hindered, and it is sufficient that a process used by a person skilled in the art for each application be followed.

### EXAMPLES

The present invention is described in detail by means of the examples shown below, but the scope of the present invention is not limited thereto.

### Example 1

### Production of Phosphatidyl Choline

A squeezed fluid (3 tons) was obtained by squeezing Antarctic krill (10 tons) caught in the Antarctic Ocean from February to November 2009 in a meat and bone separator (BADDER 605, manufactured by BADDER) immediately after being caught. This squeezed fluid was transferred to a stainless steel tank 800 kg at a time, and was heated by directly injecting water vapor of a temperature of 140° C. The heating was stopped in approximately 60 minutes of heating when it was confirmed that a temperature had reached 85° C. A valve at the bottom of the tank was opened and the liquid component was removed by allowing it to naturally drip through a mesh having a size of 2 mm. The solid content (thermal coagulum) was cleaned by showering with an amount of water equal thereto. Then, the solid content was transferred to aluminum trays 12 kg at a time and subjected to rapid freezing in a contact freezer. A total weight of the obtained coagulum was 2.25 tons.

A frozen product (1 ton) was introduced into water (3,000 liters) and heated while stirring until a temperature reached 65° C., and was held at 65° C. for 10 minutes. The water was strained using a 24 mesh nylon and the solid content was introduced to 3,000 liters of water (20° C.). After stirring for 15 minutes, the mixture was strained using a 24 mesh nylon. Then, the strained mixture was placed in a dewatering centrifuge (Centrifuge O-30, manufactured by Tanabe Willtec Inc.; 15 seconds), and a solid content was obtained having a moisture content of approximately 73%. 0.3% of tocopherol was added to this solid content. The resulting mixture was blended thoroughly using a mixer, dried for 3.2 hours by means of hot air drying at a temperature of from 70 to 75° C., and a cleaned and dried product (170 kg) was obtained. Other frozen products were processed in the same manner.

99% of ethanol (1,200 liters) was added to the cleaned and dried product (300 kg), and the resulting mixture was heated to 60° C. and mixed for two hours. Thereafter, a liquid extract A and a lees extract A were obtained by solid-liquid separation via natural dripping using a 100 mesh nylon. 99% of ethanol (800 liters) was added to the lees extract A. After heating to 60° C. and mixing for two hours, the resulting mixture was solid-liquid separated using a 100 mesh nylon, and a liquid extract B and a lees extract B were obtained. 99% of ethanol (700 liters) was added to the lees extract B. After heating to 60° C. and mixing for two hours, the resulting mixture was solid-liquid separated using a 100 mesh nylon, and a liquid extract C and a lees extract C were obtained. A combined weight of the liquid extracts A, B, and C was 2,021 kg. This combination was concentrated in vacuo at a temperature of 60° C. or less, the ethanol and water were removed, and a lipid extract (145.0 kg) was obtained. Components of the obtained lipid extract and a composition of the fatty acid are shown in Table 1 and Table 2.

TABLE 1

| | | Lipid extract |
|---|---|---|
| Moisture content | (%) | 0.48 |
| Ethanol | (%) | 0.42 |
| Phospholipid | (%) | 46.9 |
| Acid number | | 4.3 |
| Peroxide value | (meq/kg) | 0.1 |
| Astaxanthin | (ppm) | 343 |

TABLE 2

| | | Lipid extract |
|---|---|---|
| Fatty acid | C14:0 | 11.8 |
| composition | C16:0 | 22.6 |
| (%) | C18:1 | 18.5 |
| | C18:2 | 1.5 |
| | C18:3 | 0.8 |
| | C18:4 | 2.3 |
| | EPA | 14.8 |
| | DHA | 6.9 |

The lipid extract was adsorbed on a silica gel (Microsphere gel manufactured by Asahi Glass Co., Ltd.; Model: M.S. GEL SIL; 300 g) column. After adding chloroform to the column and rinsing off the neutral lipids, a phospholipid fraction (0.228 g) was collected by adding methanol. A lipid content in 10 g of the dried product of the thermal coagulum was 4.72 g.

The lipid component was separated by subjecting the phospholipid fraction to thin layer chromatography using a developing solvent containing chloroform, methanol, and water at a ratio of 65:25:4. Lipid components were quantitatively analyzed using a thin layer automatic detecting device (Model: Iatroscan™ MK-6, manufactured by Mitsubishi Kagaku Iatron, Inc.). As a result, it was discovered that the phospholipid fraction included phosphatidyl choline (96 wt %) and phosphatidylethanolamine (4 wt %).

The fatty acid in the phospholipid fraction was methyl-esterized in boron trifluoride and was subjected to gas chromatography set to the following parameters. Thereby, a fatty acid composition was analyzed.

Gas chromatography: Model: 6890N, manufactured by Agilent Technologies

Column: DB-WAX, (Model 122-7032, manufactured by J&W Scientific)

Carrier gas: Helium

Detector: Hydrogen ionization detector

The results of the analysis are shown below in Table 3.

TABLE 3

| | Content in the composition |
|---|---|
| PC content | 96 wt % |
| EPA content | 29.7 wt % |
| DHA content | 12.1 wt % |

The composition was used in the following experiments as a PC-containing composition.

### Example 2

### Production of Phosphatidylserine-Containing Composition

After adding L-serine (200 g) to a sodium acetate buffer (pH 5.6, 400 ml) and then adding phospholipase D (4000 unit/g, 2 g) of actinomycete origin, the serine was completely dissolved by mixing at 40° C. The solution was combined with astaxanthin (340 ppm) and ethyl acetate (500 ml) in which a phospholipid of krill origin (100 g) containing PC (35 wt %) was dissolved; and reacted for 24 hours at 40° C. while mixing at 200 rpm.

After completion of the reacting, the reaction solution was allowed to stand and a top layer that separated was collected. The top layer was washed three times with water in order to remove the residual serine and enzyme. By concentrating the solvent layer, a composition (85.2 g, yield with respect to the phospholipid: 85.2%) containing PS was obtained. Results of analyzing the content of the PS, EPA, and DHA in the composition are shown below in Table 4.

TABLE 4

| | Content in the composition |
|---|---|
| PS content | 30.5 wt % |
| EPA content | 15.4 wt % |
| DHA content | 7.0 wt % |

### Example 3

### Production of a Phosphatidylserine-Containing Composition

After adding L-serine (200 g) to a sodium acetate buffer (pH 5.6, 400 ml) and then adding phospholipase D (4,000 unit/g, 2 g) of actinomycete origin, the serine was completely dissolved by mixing at 40° C. The solution was combined with astaxanthin (450 ppm) and ethyl acetate (500 ml) in which a phospholipid of krill origin (100 g) containing PC (55 wt %) was dissolved; and reacted for 24 hours at 40° C. while mixing at 200 rpm.

After completion of the reacting, the reaction solution was allowed to stand and a top layer that separated was collected. The top layer was washed three times with water in order to remove the residual serine and enzyme. By concentrating the solvent layer, a PS-containing composition (85.9 g, yield with respect to the phospholipid: 85.9%) was obtained. Upon analysis using HPLC, a PS content in the composition was found to be 50.1 wt %.

### Example 4

### Production of a Phosphatidylserine-Containing Composition

Neutral lipids were removed by washing a phospholipid of krill origin (150 g) containing astaxanthin (340 ppm) and PC (35 wt %) with acetone. The phospholipid (50 g) obtained by concentrating the precipitate was dissolved in ethyl acetate (500 ml).

L-serine (200 g) and a sodium acetate buffer (pH 5.6; 400 ml) were added, and then phospholipase D (4000 unit/g, 2 g) of actinomycete origin was added to the ethyl acetate and reacted for 24 hours at 40° C. while mixing at 200 rpm.

After allowing the reaction solution to stand, the top layer was collected and washed three times with water to remove the residual serine and enzyme. The solution layer was concentrated, acetone was added, and then filtering was performed. The obtained liquid was dried under reduced pressure and a PS-containing composition powder (35.1 g, yield: 70.2%) was obtained. Results of analyzing the content of the PS, EPA, and DHA in the composition are shown below in Table 5.

TABLE 5

| | Content in the composition |
|---|---|
| PS | 90.5 wt % |
| EPA | 32.0 wt % |
| DHA | 14.4 wt % |

### Test Example 1

### Sexual Function Improvement Test

The PC-containing composition and the PS-containing composition, which were prepared by the methods according to Examples 1 to 4, were continuously orally administered to a male mouse of declined function and advanced age, and sexual function improvement effectiveness was evaluated.

### (1) Test Animal

A specific pathogen free (SPF) imprinting control region (ICR) mouse was used. A 10 months old male mouse was raised for use as the mouse having declined function and a 3 months old male mouse was raised as the mouse having normal function. After raising for 5 weeks, these mice were used in the mating experiments. A 3 months old female mouse was used for mating with each group of male mice.

The mice were raised in solidarity according under the following conditions.

Temperature: Room temperature (22 to 24° C.);

Relative humidity: 40 to 70%;

Photoperiod: 12 hours (light);

Animal feed: SPF feed, free feeding;

Drinking water: Sterile water, free drinking

After acclimating the purchased mice for three days according to the following raising conditions, the mice were divided randomly into the following groups (Table 6).

TABLE 6

| | | | | | Age at time of mating | |
|---|---|---|---|---|---|---|
| | | | | Mating | 3 months and | 10 months and |
| Administration group | | Dose | | mouse | 5 weeks | 5 weeks |
| Normal | (Base) | Physiological | | ♀ 12 | ♂ 12 | |
| comparison group | | saline | | | | |
| Advanced age | (Control) | Physiological | | ♀ 12 | | ♂ 12 |
| comparison group | | saline | | | | |
| K-PC low dose group | (K-PCL) | 10 | mg/kg/day | ♀ 12 | | ♂ 12 |
| K-PC middle dose group | (K-PCM) | 100 | mg/kg/day | ♀ 12 | | ♂ 12 |
| K-PC high dose group | (K-PCH) | 1,000 | mg/kg/day | ♀ 12 | | ♂ 12 |
| K-PS low dose group | (K-PSL) | 10 | mg/kg/day | ♀ 12 | | ♂ 12 |
| K-PS middle dose group | (K-PSM) | 100 | mg/kg/day | ♀ 12 | | ♂ 12 |
| K-PS high dose group | (K-PSH) | 1,000 | mg/kg/day | ♀ 12 | | ♂ 12 |

| | | | |
|---|---|---|---|
| Total | 96 | 12 | 96 |

In the table, the numbers represent the number of mice, ♂ represents males, and ♀ represents female

### (2) Preparation of Test Samples

The PC-containing composition and the PS-containing composition were each diluted with MCT (Medium Chain Triglycerides) and solutions of 2 mg/ml, 20 mg/ml, and 200 mg/ml were prepared.

### (3) Administration of Test Samples

The test samples were orally administered to the male mice using a feeding tube. The test samples were administered at a rate of one time per day for a duration of five weeks. Doses of the test samples were as follows: PCL and PSL (10 mg/kg), PCM and PSM (100 mg/kg), PCH and PSH (1,000 mg/kg). Physiological saline was administered to the normal comparison group and the advanced age comparison group in lieu of the PC or PS. A dose of 0.15 ml/mouse (estimated body weight of the mouse: about 30 g) of the physiological saline was administered to the normal comparison group mice, and a dose of 0.20 ml/mouse (estimated body weight of the mouse: about 40 g) of the physiological saline was administered to the advanced age comparison group mice.

### (4) Mating Test

48 hours prior to starting the mating test, each female mating mouse was injected with 20 mM of estradiol benzoate, and then 4 hours prior to starting the mating test, each female mating mouse was injected with 500 mM of progesterone.

The mating test began with the introduction of one of these female mice into each of the cages where the male mice were being raised. Behavior of the male mouse was observed for 20 minutes from the start of the test and evaluated according to the following criteria (1) to (5).

Furthermore, the testis (+the epididymis) and seminal vesicles (+the prostate) of the male mouse were extracted and a weight thereof was measured (evaluation criterion 6).

Evaluation criteria:

1. Incubation period of mounting

2. Incubation period of copulation (ejaculation)

3. Mounting frequency

4. Copulation frequency

5. Ratio of mounting frequency to copulation frequency (copulation ratio)

6. Weight of the testis (+the epididymis) and the seminal vesicles (+the prostate)

### (5) Results

In the experiments a clear significant difference was observed for all evaluation criteria between the advanced age comparison group (control) and the normal comparison group (base).

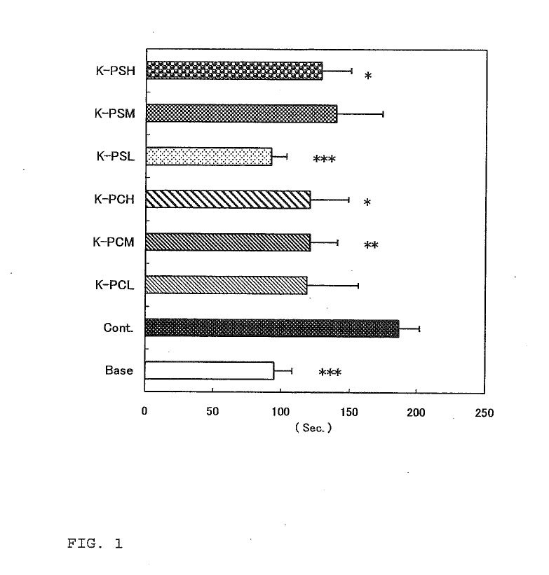

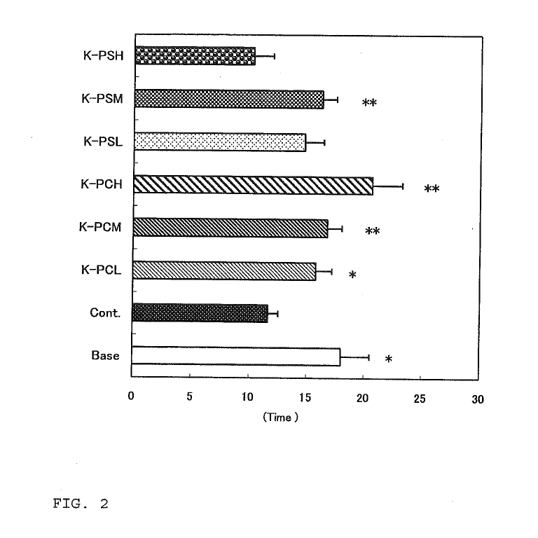

### (5-1) Incubation Period of Mounting and Mounting Frequency (FIG. 1 and FIG. 2)

For all of the administration groups, it was shown that the incubation period of mounting of the male animals having advanced age was significantly shorter than the incubation period of mounting of the comparison group of animals being likewise of advanced age (control), and that the incubation period of mounting of each of the administration groups approached the incubation time of mounting of the young normal comparison group (base). Particularly, the PSH, PSL, PCH, and PCM groups displayed prominent improvement effectiveness.

Additionally, it was shown that for most of the administration groups, not only the incubation time of mounting, but also the mounting frequency of the male animals having advanced age increased greatly compared to the comparison group of animals being likewise of advanced age (control), and that a degree of increase thereof reached levels equivalent to the mounting frequency of the young normal comparison group (base). Among these, the PSM, PCH, PCM, and PCL groups displayed significant improvement effectiveness.

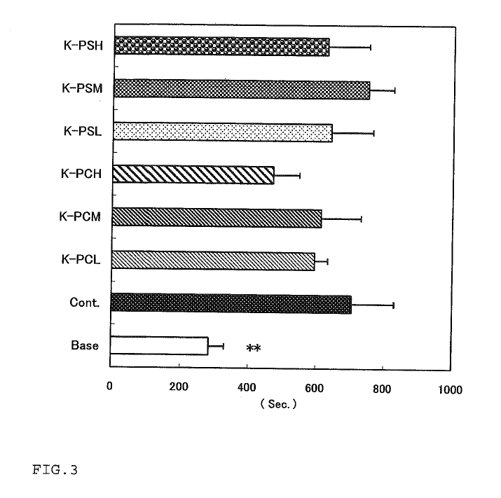

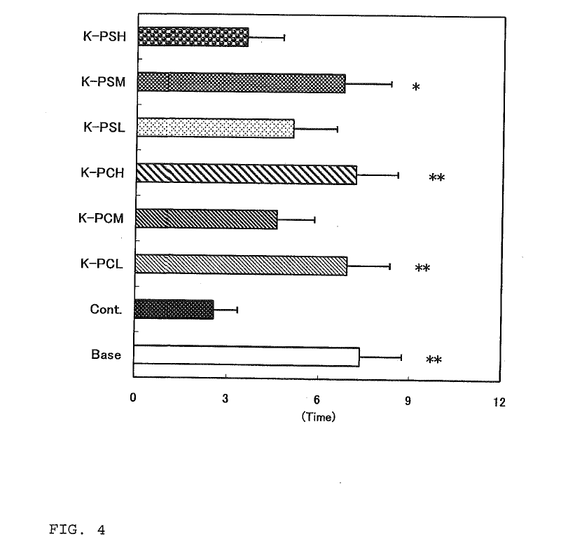

### (5-2) Incubation Period of Copulation (Ejaculation) and Copulation Frequency (FIG. 3 and FIG. 4)

On the other hand, when measuring the incubation period of copulation and copulation frequency of the male animals having advanced age, is was observed that, compared to the comparison group of animals being likewise of advanced age (control), the incubation period of copulation had a tendency to shorten due to the administration of PC and PS. Moreover, a prominent improvement effect in copulation (frequency) in the PSM, PCH and PCL administration groups was confirmed (FIG. 4). Note that it was found that this improvement effect in the copulation of male animals having advanced age was comparable with the young normal comparison group (base).

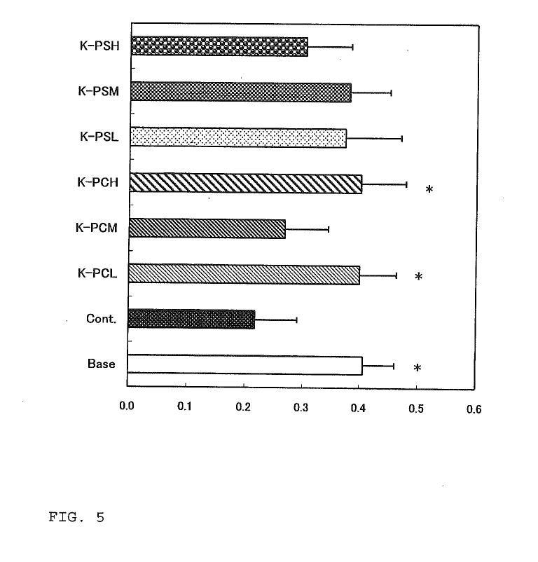

### (5-3) Copulation Ratio (FIG. 5)

For all of the administration groups, it was shown that the copulation ratio of the male animals having advanced age was significantly increased compared to the copulation ratio of the comparison group of animals being likewise of advanced age (control), and that the copulation ratio of each of the administration groups approached the copulation ratio of the young normal comparison group (base). In the low administration groups and the high administration groups, more certain improvement effectiveness was shown in the PC administration groups than in the PS administration groups.

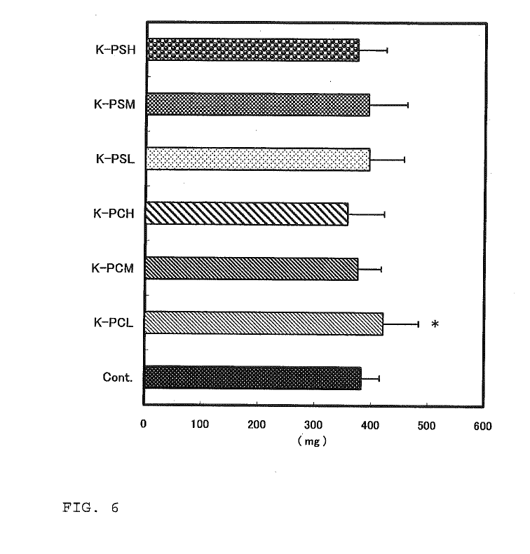

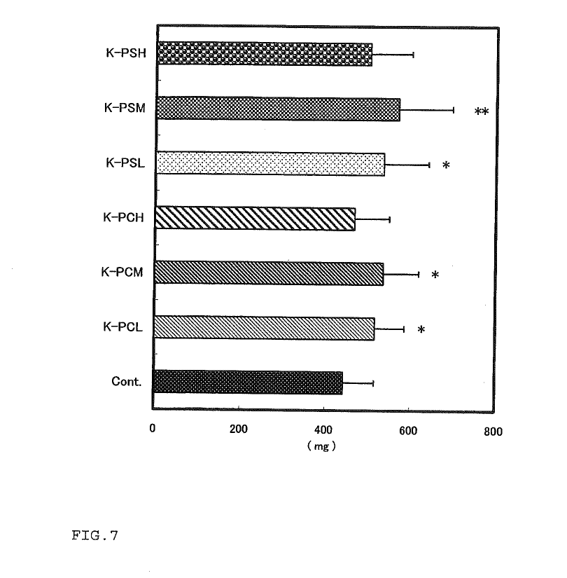

### (5-4) Weight of the Testis (+the Epididymis) and Weight of the Seminal Vesicles (+the Prostate) (FIG. 6 and FIG. 7)

When measuring the weight of the reproductive organs of the male animals having advanced age, it was confirmed that, compared to the comparison group of animals being likewise of advanced age (control), the weight of the testis (+the epididymis) was substantially unchanged, but that the weight of the seminal vesicles (+the prostate) increased prominently due to the administration of the PC and PS.

According to the results described above, with the male animals to which the sexual function improving agent of the present invention was administered, compared to the male animals of declined function and advanced age to which a sexual function improving agent was not administered, a reduction in the incubation period of mounting and an increase in mounting frequency, a reduction in the incubation period of copulation and an increase in the copulation frequency, and an increase in the copulation ratio occurred. Thereby, it was shown that the sexual behavior of male animals is vitalized. Furthermore, because the sexual function improving agent of the present invention increases the weight of the reproductive organs of the male animal to which it is administered, particularly the seminal vesicles (+the prostate), it is implied that spermatogenesis is vitalized.

However, while these effects are comparable with young animals having normal sexual function, it is clear that effects surpassing young animals having normal sexual function are not obtained.

Therefore, it is implied that while the sexual function improving agent of the present invention can improve normal sexual function by restoring the declined sexual function of male animals, the sexual function improving agent of the present invention does not have an effect of invigorating beyond normal sexual function. Such effects differ from existing sexual stamina enhancers that have invigorating effects and are considered to be based on the nutritional function of the lipid, particularly on the lipid including a highly unsaturated fatty acid in the fatty acid part. This effect was entirely unexpected.

### Test Example 2

### (1) Test Animal

A specific pathogen free (SPF) imprinting control region (ICR) mouse was used for the test. Eleven months old male mice were raised for use as the control mice and each group. Three months old male mice were raised as the mice having normal function. Three months old female mice were used for mating with each group of male mice.

### (2) Raising Conditions for Mice

The mice were raised in solidarity under the following conditions.

Temperature: Room temperature (22 to 24° C.); Relative humidity: 40 to 70%;

Artificial light: light and dark condition for every 12 hours;

Animal feed: SPF feed;

Drinking water: Sterile water.

### (3) Grouping

After the purchased mice were acclimatized for three days, the mice were divided randomly into the following groups, based on the body weight of the mice

TABLE 7
Grouping of mice

| | | | | Age at time of mating | |
|---|---|---|---|---|---|
| | | | | 3 | 11 |
| Animal group | | Dose | | months | months |
| Normal | (Base) | Physiological | | ♂ 12 + | |
| comparison group | | saline | | ♀ 12 | |
| Advanced age | (Control) | Physiological | | ♀ 12 | ♂ 12 |
| comparison group | | saline | | | |
| K-PC low | (K-PCL) | 10 | mg/kg/day | ♀ 12 | ♂ 12 |
| dose group | | | | | |
| K-PC middle | (K-PCM) | 100 | mg/kg/day | ♀ 12 | ♂ 12 |
| dose group | | | | | |
| K-PC high | (K-PCH) | 1,000 | mg/kg/day | ♀ 12 | ♂ 12 |
| dose group | | | | | |

| | | |
|---|---|---|
| Total | ♂ 12, | ♂ 84 |
| | ♀ 96 | |

### (4) Test Samples

The following samples were used for each animal group.

K-PC group: krill oil, which can also be called as krill phosphatidylcholine or krill choline;

and

Control group: physiological saline.

### (5) Preparation of Test Samples

The K-PC was each diluted with MCT and to prepare solutions of 2 mg/ml, 20 mg/ml, and 200 mg/ml. These samples were used as the test samples.

### (6) Administration of Test Samples

The test samples were orally administered to the male mice using a feeding tube at a rate of one time per day.

(7) Dose and Term For Administration

The test samples were administered to animals at 10 mg/kg for low dose groups, 100 mg/kg for middle dose groups, and 1,000 mg/kg for high dose groups, namely, the dose is dependent on the body weight of individual animals. Physiological saline was administered to the animals in normal comparison group and the animals in advanced age comparison group. A dose of 0.15 ml/mouse (estimated body weight of the mouse: about 30 g) of the physiological saline was administered to the animals in normal comparison group mice, and a dose of 0.20 ml/mouse (estimated body weight of the mouse: about 40 g) of the physiological saline was administered to the animals in advanced age comparison group mice.

### (8) Mating Test

Forty eight hours prior to starting the mating test, each female mating mouse was injected with 20 μM of estradiol benzoate, and then 4 hours prior to starting the mating test, each female mating mouse was injected with 500 μM of progesterone.

The mating test was started by transferring one of these female mice into each of the cages where the male mouse was being raised. Behavior of the male mice was observed for 20 minutes from the start of the test.
Testis (+the epididymis) and seminal vesicles (+the prostate) were harvested from the animals. Weights of the testis (+the epididymis) and the seminal vesicles (+the prostate) were measured. Pathological sections of spermary (testis) were prepared.

### (9) Items Evaluated

In the mating test, the following items were evaluated:

Incubation period of mounting, which means time required to perform first mounting after the start of the mating test;

Incubation period of copulation (ejaculation), which means time required to perform first copulation after the start of the mating test;

Mounting frequency, which means number of times of mounting performed during the test time (20 minutes);

Copulation frequency, which means number of times of copulation performed during the test time (20 minutes);

Ratio of mounting frequency to copulation frequency (copulation ratio), which means the number of times of copulation divided by the number of times of mounting;

Weights and weight ratio of the testis (+the epididymis) and the seminal vesicles (+the prostate);

Pathological observation on convoluted seminiferous tubules in tissues of spermary (testis);

### (10) Results

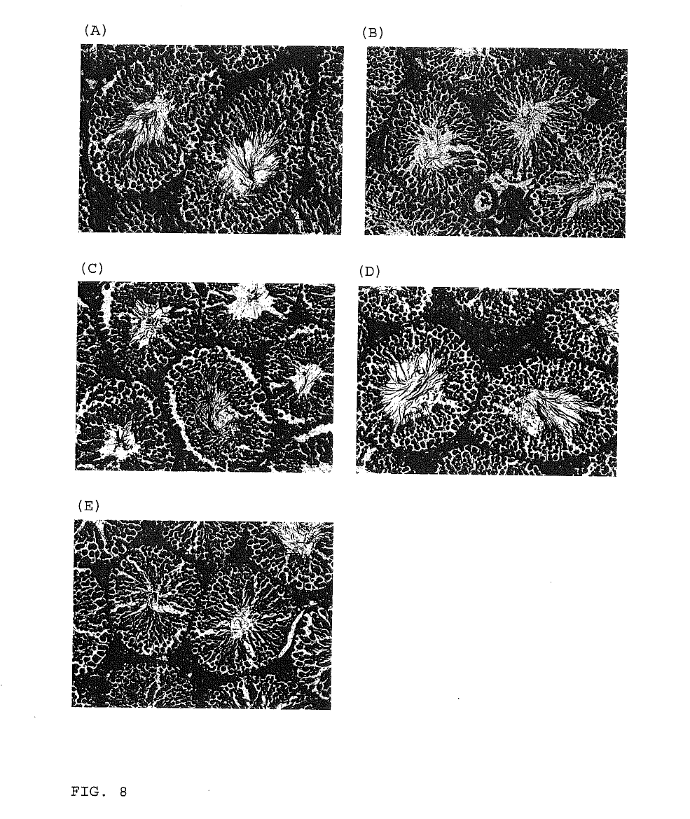

The state of convoluted seminiferous tubules harvested from each test group was observed by using pathological sections of spermary (testis). The observation showed that spermiogenesis of advanced age mice was reduced (FIG. 8(B)) and that the regression was improved by administration of K-PC (FIGS. 8(C) to (E)).

In normal mice, the process of spermatogenesis was clearly observed in convoluted seminiferous tubules (FIG. 8(A)). In addition, in normal mice, the cells in each of the stages of the spermatogegesis: spermatogenia, primary spermatocyte, secondary spermatocyte, spermatid, and sperm, were clearly observed in the order from outside to center of convoluted seminiferous tubules. Further, in normal mice, many sperm, which were generated, were also observed.

On the other hand, in advanced age mice, convoluted seminiferous tubules were atrophied. In addition, in advanced age mice, maturation division in each stage of spermatogenesis was ambiguous, and numbers of sperm were very few in convoluted seminiferous tubules (FIG.8(B)).

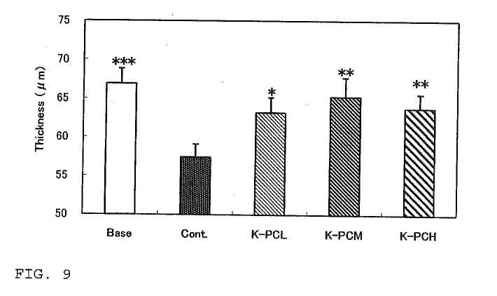

The administration of K-PC improved the atrophy to induce a number of sperm in convoluted seminiferous tubules of advanced age mice (FIGS. 8(C) for K-PCL, (D) for K-PCM, and (E) for K-PCH)). In addition, results, which explain the phenomena of atrophy of convoluted seminiferous tubules in advanced age mice as well as the effect of improvement of the atrophied convoluted seminiferous tubules by administering K-PC, were obtained by measuring a thickness of the wall of convoluted seminiferous tubules (FIG. 9).

### Test Example 3

### (1) Test Animal

A specific pathogen free (SPF) imprinting control region (ICR) mouse was used for the test. Eleven months old male mice were raised for use as the control mice and each group. Three months old male mice were raised as the mice having normal function. Three months old female mice were used for mating with each group of male mice.

### (2) Raising Conditions For Mice

The mice were raised in solidarity under the following conditions.

Temperature: Room temperature (22 to 25° C.);

Relative humidity: 40 to 70%;

Artificial light: light and dark condition for every 12 hours;

Animal feed: SPF feed;

Drinking water: Sterile water.

### (3) Grouping

After the purchased mice were acclimatized for three days, the mice were divided randomly into the following groups, based on the body weight of mice (Table 8).

TABLE 8
Grouping of mice

| | | | | Age at time of mating | |
|---|---|---|---|---|---|
| | | | | 3 | 11 |
| Animal group | | Dose | | months | months |
| Normal | (Base) | Physiological | | ♂ 12 + | |
| comparison group | | saline | | ♀ 12 | |
| Advanced age | (Control) | Physiological | | ♀ 12 | ♂ 12 |
| comparison group | | saline | | | |
| K-PC low | (K-PCL) | 10 | mg/kg/day | ♀ 12 | ♂ 12 |
| dose group | | | | | |
| K-PC middle | (K-PCM) | 100 | mg/kg/day | ♀ 12 | ♂ 12 |
| dose group | | | | | |

| | | |
|---|---|---|
| Total | ♂ 12, | ♂ 36 |
| | ♀ 48 | |

### (4) Test Samples

The following samples were used for each animal group.

K-PC group: krill oil, which can also be called as krill phosphatidylcholine or krill choline; and

Control group: physiological saline.

### (5) Preparation of Test Samples

The K-PC was diluted with MCT to prepare solutions of 2 mg/ml and 20 mg/ml of K-PC. These samples were used as the test samples.

### (6) Administration of Test Samples

The test samples were orally administered to the male mice using a feeding tube at a rate of one time per day.

### (7) Dose and Term For Administration

The test samples were administered to animals at 10 mg/kg for low dose groups, and 100 mg/kg for middle dose groups, namely, the dose is dependent on the body weight of individual animals. The volume of each sample was 5 ml/kg.

A dose of 0.20 ml/mouse (estimated body weight of the mouse: about 40 g) of the physiological saline was administered to the mice in normal group and the mice in advanced age comparison group.

### (8) Mating Test

Forty eight hours prior to starting the mating test, each female mating mouse was injected with 20 μM of estradiol benzoate, and then 4 hours prior to starting the mating test, each female mating mouse was injected with 500 μM of progesterone.

The mating test was started by transferring one of these female mice into each of the cages where a male mouse was being raised. Behavior of the male mice was observed for 30 minutes from the start of the test.

Within 24 hours after the mating test, the test animals were euthanized to harvest organs relating to reproductive function, such as spermary (testis), parorchis (epididymis), and prostate. Weights of these organs were measured.

One of the cauda epididymis, which were harvested from the animal, was divided in capacitation medium (Medium 199) at 37° C. After incubation for 5 minutes, a stock solution of sperm was obtained. By subjecting the stock solution of sperm to TOX IVOS (Hamilton Thorne Research), items relating to evaluation on the state of sperm such as sperm viability, motile percent of sperm, and progressive percent of sperm were determined.

### (9) Items Evaluated

In the mating test, the following items were evaluated:

Incubation period of mounting, which means time required to perform first mounting after the start of the mating test;

Incubation period of copulation (ejaculation), which means time required to perform first copulation after the start of the mating test;

Mounting frequency, which means number of times of mounting performed during the test time (30 minutes);

Copulation frequency, which means number of times of copulation performed during the test time (30 minutes);

Ratio of mounting frequency to copulation frequency (copulation ratio), which means the number of times of copulation divided by the number of times of mounting;

Weights of spermary, parorchis, and prostate;

Determination of total count of sperm, motile percent of sperm, progressive percent of sperm, path velocity (VAP), and progressive velocity (VSL).

### (10) Results

By this experiment, the following results were obtained.

Although no significance was observed in data among animal groups, observation on behavior of male mice showed phenomena of reduced sexual function in advanced age mice and improvement of the reduced sexual function by administration of K-PC (Table 9).

TABLE 9
Observation on behavior of male mice in each group

| | | Incubation | Incubation | | | Ratio of |
|---|---|---|---|---|---|---|
| | | period of | period of | Mounting | Copulation | mounting |
| Aminal | Dose | mounting | copulation | frequency | frequency | frequency |
| group | (mg/kg) | (Sec.) | (Sec.) | (time) | (time) | (%) |
| Base | 0 | 210.8 ± 40.3 | 454.7 ± 82.3 | 25.9 ± 2.8 | 12.3 ± 1.7 | 47.2 ± 4.0 |
| Cont. | 0 | 365.6 ± 90.6 | 687.2 ± 160.6 | 21.9 ± .2.6 | 8.1 ± 1.3 | 36.8 ± 4.2 |
| K-PCL | 10 | 241.1 ± 75.1 | 582.9 ± 142.8 | 25.0 ± 3.3 | 9.9 ± 1.7 | 38.9 ± 5.0 |
| K-PCM | 100 | 295.1 ± 79.1 | 632.1 ± 79.2 | 25.8 ± 4.3 | 8.7 ± 1.2 | 37.6 ± 5.4 |

(Mean ± SE)

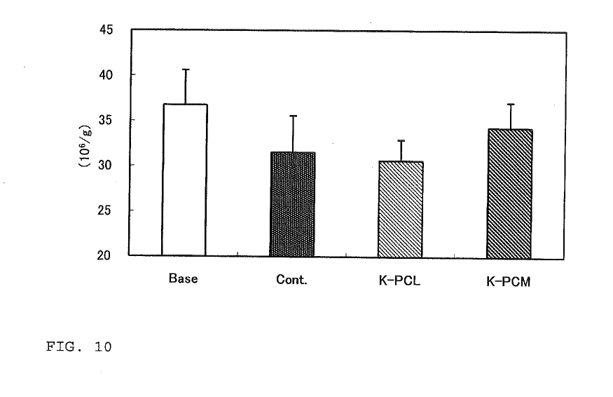

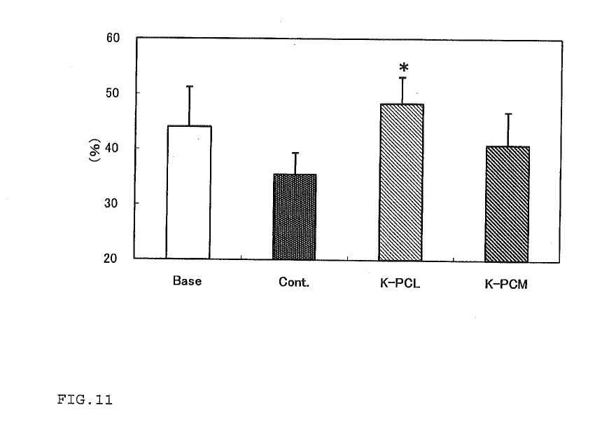

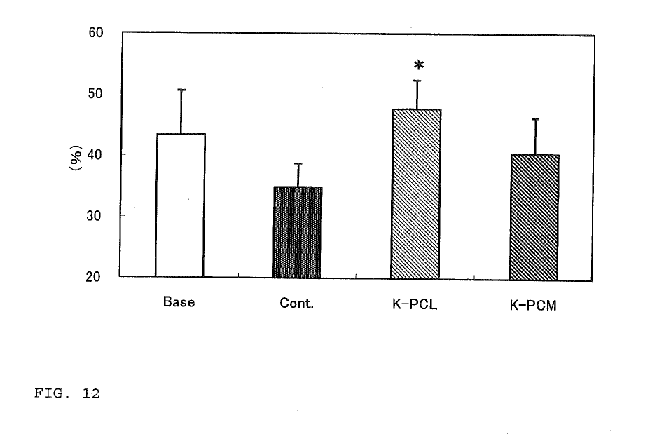

By determining the factors relating to the state of sperm (Table 10), such as total count of sperm (FIG. 10), motile percent of sperm (FIG. 11), and progressive percent of sperm (FIG. 12), it was observed that the state of spermiogenesis as well as the sperm activity was reduced in advanced age mice. The administration of K-PC improved such reduction; especially, it significantly improved the sperm activity.

TABLE 10
Parameters of sperm obtained from mice in each group

| | | Total | Motile percent | Progressive | | Average |
|---|---|---|---|---|---|---|
| | | count of | of sperm | percent of sperm | Average path | progressive |
| | | sperm | (%) | (%) | velocity | velocity |
| Animal | Dose | (10⁶/g) | Motile | Progressive | (μm/Sec.) | (μm/Sec.) |
| group | (mg/kg) | T. Count | Precent | Precent | VAP | VSL |
| Base | 0 | 36.7 ± 3.9 | 44.0 ± 7.2 | 43.4 ± 7.2 | 100.5 ± 7.0 | 67.6 ± 4.9 |
| Cont. | 0 | 31.5 ± 4.0 | 35.5 ± 3.9 | 34.9 ± .3.8 | 88.9 ± 4.6 | 60.1 ± 4.6 |
| K-PCL | 10 | 30.6 ± 2.3 | 48.3 ± 4.8* | 47.8 ± 4.7* | 100.2 ± 5.6 | 68.0 ± 4.7 |
| K-PCM | 100 | 34.3 ± 2.8 | 40.8 ± 5.9 | 40.5 ± 5.8 | 93.8 ± 6.1 | 63.0 ± 5.1 |

T-test vs Control,
*p < 0.05
(Mean ± SE)

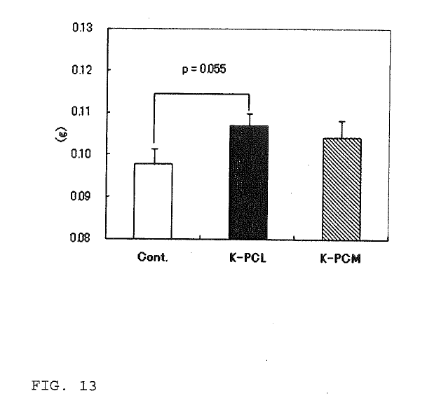

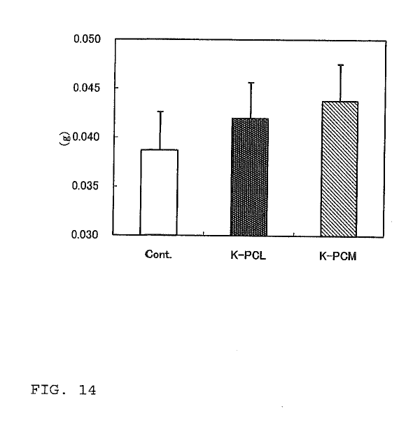

Further, by measuring weights of spermary, which produces sperm, parorchis, which stores sperm, and prostate, which produces prostatic fluid and provide sperm with nutrients, it was showed that weights of parorchis (FIG. 13) and prostate (FIG. 14) in the group of K-PC were heavier than that in the group of advanced age mice.

In light of the foregoing, it is suggested that the effect of krill phospholipids on improvement of sexual function is not based on the enhancement of sexual function but is based on the improvement of the capabilities of spermiogenesis, maintenance of sperm count, motile percent of sperm, progressive percent of sperm, path velocity, progressive velocity, which are reduced by aging. Namely, krill phospholipids can be used for improving the capabilities of spermiogenesis, maintenance of sperm count, motile percent of sperm, progressive percent of sperm, path velocity and progressive velocity, which are reduced by aging.

### Test Example 4

After administering the sexual function improving agent of the present invention to mice, sex hormone concentration in serum was determined.

(1) Mating test was carried out in accordance with the experimental conditions described in Test Example 1. After the mating test, 0.5 to 0.8 ml of blood was collected from the animals, and subjected to centrifugation at a temperature of 4° C. or lower, at 3,000 rpm/minute, for 15 minutes. The supernatant, serum was collected and used for measuring its biochemical indices as follows.

### (2) Measurement of Sex Hormone Concentration in the Serum

Concentration of Inhibin B and Follicle-Stimulating Hormone in the serum was measured for each administration group after the experiment completed Inhibin B (InB) concentration was measured using a commercial measuring kit (Beijing Sino-uk Institute of Biological Technology (Beijing, China)) under the instruction of the manual. The Follicle-Stimulating Hormone (FSH) concentration was measured using the Follicle-Stimulating Hormone Radioimmunoassay (RIA) Kit (Beijing Sino-uk Institute of Biological Technology (Beijing, China)) under the instruction of the manual.

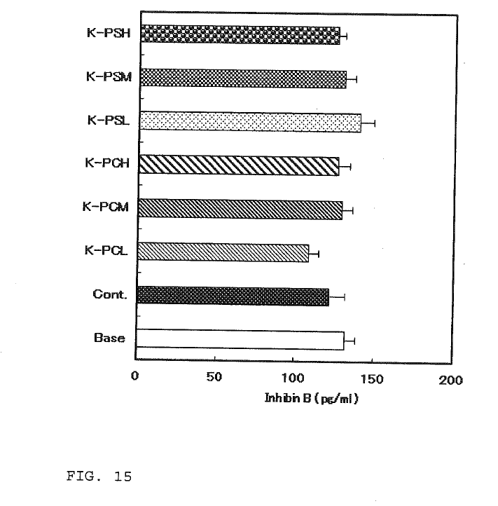

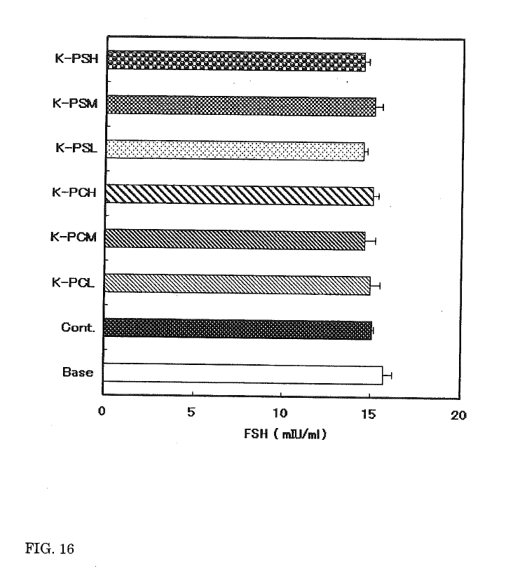

### (3) Results (FIG. 15 and FIG. 16)

The results explicitly showed that no difference exists between the different administration groups.

Those show that the effects of the sexual function improving agent of the present invention are not brought about by the increase in sex hormones, such as Inhibin B and Follicle-Stimulating Hormone.

### Test Example 5

After administering the sexual function improving agent of the present invention to mice, reproductive function of the mice was evaluated. Fish oil (F-Oil), soybean phospholipid (S-PC) and MCT (for control) were continuously administered orally to an aged mouse to evaluate their effects in improving the reproductive function.

### (1) Test Animal

Specific pathogen free (SPF) imprinting control region (ICR) mice were used. Twelve months old male mice were raised for use as mice having declined function. After raising the mice for 4 weeks, the mice were used for evaluating reproductive functions.

The mice were raised in solidarity under the following conditions.

Temperature: Room temperature (22 to 25° C.);

Relative humidity: 40 to 70%;

Photoperiod: 12 hours (light)

Animal feed: SPF feed, free feeding;

Drinking water: Sterile water, free drinking

After acclimating the purchased mice for three days according to the above raising conditions, the mice were divided randomly into the following groups (Table 11).

TABLE 11

| Animal group | | Dose | 13 months old |
|---|---|---|---|
| MCT group | (Control) | 100 mg/kg/day | ♂ 15 |
| F-Oil group | (F-Oil) | 100 mg/kg/day | ♂ 15 |
| S-PC group | (S-PC) | 100 mg/kg/day | ♂ 15 |
| F-Oil + S-PC group | (F + S) | (50 mg + 50 mg)/ | ♂ 15 |
| | | kg/day | |

### (2) Preparation of Test Samples

Fish-oil (triglycerides as lipid 99.0%(w/w), tocopherol 1.0%(w/w), water 0.0%(w/w) with fatty acid composition of EPA 28.8%(w/w) and DHA 14.7%(w/w)) and soybean phospholipid (purchased from Wako Pure Chemical Industries, Ltd., Lecithin from Soybean, product number 120-00832) were each diluted with MCT and solutions of 20 mg/ml were prepared.

### (3) Administration of Test Samples

The test samples were orally administered to the male mice using a feeding tube. The test samples were administered at a rate of once per day for a duration of four weeks. Test samples were administered at 5 ml/kg according to the body weight of each animal to achieve 100 mg/kg (Middle-dose group).

### (4) Evaluation of the Reproductive Function

Within 24 hours after the mating test, the test animals were euthanized to harvest organs relating to the reproductive function, such as testis, epididymis, and prostate. Weights of these organs were measured. One of the cauda epididymis, which was harvested from the animal, was divided in the capacitation medium (Medium 199) heated to 37° C. After incubation for 5 minutes, a stock solution of sperm was obtained. By subjecting the stock solution of sperm to TOX IVOS (Hamilton Thorne Research), the following items relating to evaluation on the state of sperm such as sperm viability, motile percent of sperm, and progressive percent of sperm were determined

Evaluation Items: 1. Weights of testis, epididymis, and prostate; 2. Total count of sperms, motile percent of sperms, progressive percent of sperms.

### (5) Results

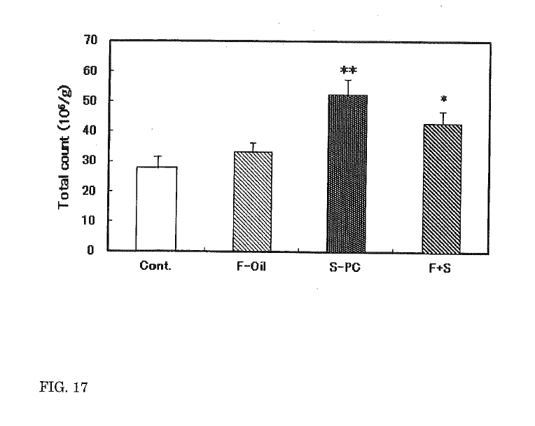

### (5-1) Total Count of Sperms (FIG. 17)

The total counts of sperms of the F-Oil group, the S-PC group, and the F+S group all increased from that of the control group. The counts of the S-PC group and the F+S group increased significantly from that of the control group. The count of the S-PC group was the highest.

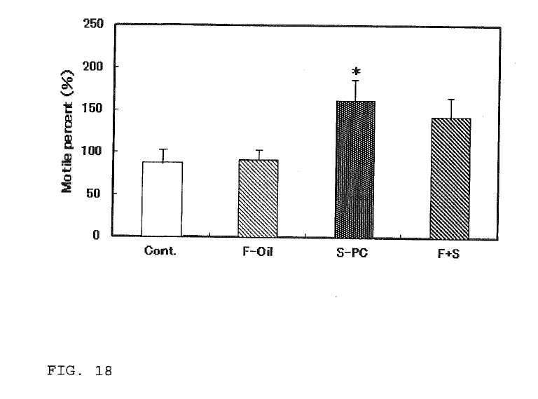

### (5-2) Motile Percent of Sperms of Each Administration Group (FIG. 18)

The motile percents of sperms of the S-PC group and the F+S group increased from that of the control group. Specifically, the motile percent of the S-PC group increased significantly.

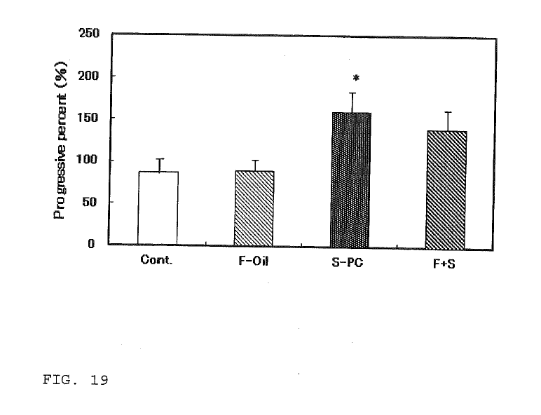

### (5-3) Progressive Percent of Sperms of Each Administration Group (FIG. 19)

The progressive percent of sperms of the S-PC group and the F+S group increased from that of the control group. Specifically, the progressive percent of the S-PC group increased significantly.

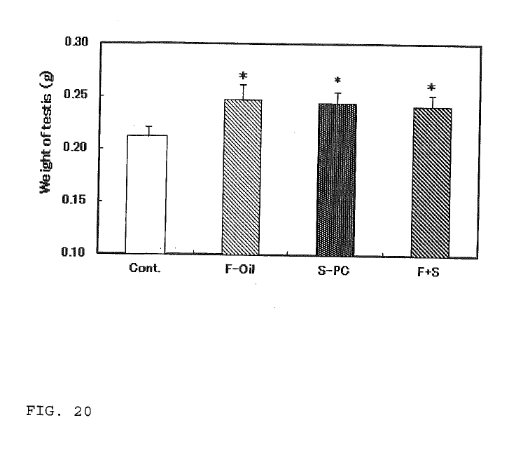

### (5-4) Weight of Testis of Each Administration Group (FIG. 20)

The weights of testes of the F-Oil group, the S-PC group, and the F+S group all increased significantly from that of the control group.

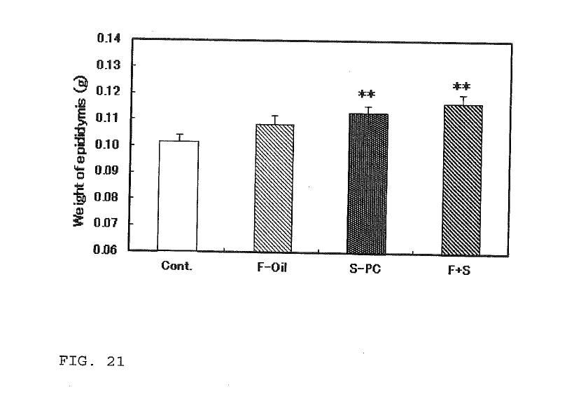

### (5-5) Weight of Epididymis of Each Administration Group (FIG. 21)

The weights of epididymides of the F-Oil group, the S-PC group, and the F+S group all increased significantly from that of the control group. The weights of the S-PC group and the F+S group increased significantly from that of the control group.

The above results explicitly show that the sexual function improving agent of the present invention, comprising phospholipid as an active ingredient, improves the reproductive function.

In light of the foregoing results of Test Examples 1 to 5, it is presumed that the effects of phospholipids including unsaturated fatty acids, such as krill phospholipid and soybean phospholipid, on improvement of sexual function is not based on the enhancement of sexual function but is based on preventing or recovering the capabilities of spermiogenesis and sperm-count maintenance of the living body, which are reduced by aging.

1.-12. (canceled)

13. A method of improving sexual function of an animal comprising orally administering to the animal in need thereof an effective amount of the sexual function improving agent comprising a phospholipid as an active ingredient, wherein the phospholipid comprises an n-3 highly unsaturated fatty acid as a constituent fatty acid.

14.-15. (canceled)

16. The method of claim 13, wherein the animal is an animal other than human.

17.-40. (canceled)

41. The method according to claim 13 comprising administering to the animal in need thereof the phospholipid at a dosage of 1 to 5000 mg/50 kg BW/day.

42. The method according to claim 13, wherein the n-3 highly unsaturated fatty acid is eicosapentaenoic acid or docosahexenoic acid.

43. The method according to claim 13, wherein the phospholipid is selected from the group consisting of phosphatidylserine, phosphatidyl choline, phosphatidylethanolamine, phosphatidic acid, phosphatidylglycerol, and phosphatidylinositol.

44. The method according to claim 13, wherein purified krill oil is used as an active ingredient.

45. The method according to claim 13, wherein krill oil is used as an active ingredient.

46. The method according to claim 44, wherein the krill oil is purified via a thermal coagulum of krill.
